(19) United States
(12) Patent Application Publication
(10) Pub. No.: US 2024/0355410 A1
(43) Pub. Date: Oct. 24, 2024

(54) METHOD AND SYSTEMS FOR A MACHINE-ASSISTED DISCOVERY OF CHONDROITINASE ABC COMPLEXES TOWARDS SUSTAINED NEURAL REGENERATION

(71) Applicants: RUTGERS, THE STATE UNIVERSITY OF NEW JERSEY, New Brunswick, NJ (US); THE TRUSTEES OF PRINCETON UNIVERSITY, Princeton, NJ (US)

(72) Inventors: Adam J. Gormley, Piscataway, NJ (US); Matthew Tamasi, Piscataway, NJ (US); Shashank Kosuri, Piscataway, NJ (US); Michael Anthony Webb, Pennington, NJ (US); Carlos Hernan Borca, Pennington, NJ (US); Roshan Anit Patel, Princeton, NJ (US)

(21) Appl. No.: 18/645,966

(22) Filed: Apr. 25, 2024

Related U.S. Application Data
(63) Continuation of application No. PCT/US22/48044, filed on Oct. 27, 2022.
(60) Provisional application No. 63/272,432, filed on Oct. 27, 2021.

(51) Int. Cl. G16B 15/20; G06F 30/27; G16B 15/30; G16B 40/20

(52) U.S. Cl. CPC G16B 15/20; G06F 30/27; G16B 15/30; G16B 40/20

(57) ABSTRACT

Disclosed are systems and methods that provide a framework for machine-assisted discovery of Chondroitinase ABC (ChABC) complexes towards sustained neural regeneration. In some embodiments, the framework may leverage a determination of a diverse set of tailor-made random copolymers that complex and stabilize ChABC at physiological temperature. The copolymer designs, which are based on chain length and/or composition of the copolymers, may be identified using an active machine learning paradigm, which involves, but is not limited to, copolymer synthesis, testing for ChABC thermostability upon copolymer complexation, Gaussian Process Regression modeling and Bayesian optimization. Copolymers are synthesized by automated PET-RAFT, and thermostability of ChABC may be assessed by retained enzyme activity (REA) after a predefined interval of hours at 37° C.

### CROSS REFERENCE TO RELATED APPLICATIONS

This application is a Continuation Application relating to and claiming the benefit of commonly-owned, co-pending PCT International Application No. PCT/US2022/048044, filed Oct. 27, 2022, which claims priority to and the benefit of U.S. Provisional Patent Application No. 63/272,432, filed Oct. 27, 2021, the contents of each of the foregoing are herein incorporated by reference in their entirety.

According to some embodiments, at least some methods and/or principles described herein may be accomplished or further detailed as described in related application PCT/US2021/031114, which is incorporated herein by its entirety.

### STATEMENT OF RIGHTS TO INVENTIONS MADE UNDER FEDERALLY SPONSORED RESEARCH

This invention was made with government support under GM138296 awarded by the National Institutes of Health. The government has certain rights in the invention.

### FIELD OF TECHNOLOGY

The present disclosure relates to the design and stabilization of proteins, and more particularly to methods and systems for machine-assisted discovery of Chondroitinase ABC complexes towards sustained neural regeneration.

### BACKGROUND OF TECHNOLOGY

Proteins may be used in many biopharmaceuticals, biological and medical applications. Biopharmaceuticals may include any pharmaceutical drug product manufactured in, extracted from, or synthesized in part from biological sources. Biopharmaceuticals may include, for example, vaccines, blood, blood components, allergenics, somatic cells, gene therapies, tissues, recombinant therapeutic protein, and living medicines used in cell therapy. They may be composed of sugars, proteins, or nucleic acids or complex combinations of these substances, or may be living cells or tissues.

Proteins, in the form of enzymes, play a significant role in many commercial and industrial due to their high catalytic potential across a wide range of substrates. For example, enzymes typically operate under precise conditions of temperature and pH. However, ex vivo conditions for using enzymes are more demanding. In most instances, these enzymes are exposed to harsh conditions such as organic solvents, heat, denaturants or acids/bases to facilitate process efficiency. However, these harsh conditions result in enzyme destabilization which necessitates continuous addition of fresh and costly enzyme to the reaction mixture.

Complex synthetic polymers may stabilize proteins such as enzymes under harsh conditions by providing a chaperone-like stabilizing shell. More recently, the use of single enzyme nanoparticles (SEN) has emerged as an attractive method for stabilizing enzymes, for example. In these cases, individual enzymes may be wrapped in a protective coating to stabilize the enzyme structure. By carefully designing this enzyme-material interface, it may be possible to provide enzyme durability in extremely unnatural environments during the polymer synthesis that the enzyme is catalyzing.

### BRIEF SUMMARY

Some embodiments, without limitation, may utilize Chondroitinase ABC (ChABC), which is an enzyme derived from Proteus Vulgaris, has shown potential for treating spinal cord injuries because of its ability to degrade certain molecules in scar tissue and promote axonal regeneration. However, it is unstable and may lose all of its activity within few hours at 37° C., necessitating the use of repeated injections or multiple infusions for days to weeks during medical treatment for treating the injuries to the central nervous system (CNS), including traumatic brain injuries (TBI) and spinal cord injuries (SCI). Typically, these infusion systems are highly invasive, infection prone, and clinically problematic to administer.

Moreover, ChABC suffers from poor thermal stability under physiological conditions. This may severely restrict its use as a viable therapeutic option for treating CNS injuries. While various techniques have been reported for ChABC stabilization, the disclosed systems and methods utilize a novel technique of using copolymers for the very first time to stabilize ChABC in artificial cerebrospinal fluid (aCSF). In some embodiments, by coupling automated polymer chemistry with active learning, several polymers have been discovered that stabilize ChABC at physiological temperature in aCSF.

In some embodiments, in general, polymers identified by active learning exhibited significantly better retained enzyme activity (REA) for ChABC compared to a large, systematic screen, and designs evidenced improvements with the acquisition of more data. For example, the instant disclosure's techniques may realize a remarkably stabilized ChABC for over 7 days at a very low concentration, as discussed in more detail below.

While polymer chemistry has been historically low throughput, recent advancements in the field of automated polymer synthesis using oxygen tolerant chemistries enable higher throughput screening of combinatorial polymer libraries with relative ease. While systematic exploration-based design yielded modest results, coupling active learning with high-throughput experimentation enables the efficient identification of unique copolymers with propensity to stabilize ChABC.

According to some embodiments, the use of the disclosed designed copolymers for thermostabilization of ChABC has shown to be competitive with or superior to other existing stabilization strategies, as evidenced from the instant disclosure. These encouraging results may enable the facilitation of glial scar degradation and promote neural tissue regeneration after injury.

According to some embodiments, described herein may be a system for machine-assisted discovery of ChABC complexes towards sustained neural regeneration. The system includes an instrumentation platform comprising at least one instrument, at least one measurement device, or both; and at least one processor, where the at least one processor is configured to: identify a set of copolymers, each copolymer comprising a chain length and composition; analyze the set of copolymers, and determining a set of features of the set of copolymers, the determined set of features at least corresponding to a chain length and composition of monomers; perform a copolymer synthesis for each of the set of copolymers based on the determined set of features; determine a copolymer complexation based on the performed copolymer synthesis; determine a thermostability of a protein based on the determined copolymer complexation; execute a regression model, the execution of the regression model being based on the thermostability of the protein; execute an optimization model, the optimization model generating a copolymer design, the copolymer design comprising at least a chain length and composition of monomers of a copolymer that enables the thermostability of the protein; and apply a selected copolymer to the protein, wherein the selected copolymer comprises a chain length and composition of monomers corresponding to the copolymer design.

According to some embodiments, a method is provided for machine-assisted discovery of ChABC complexes towards sustained neural regeneration. The method includes: identifying, by a device, a set of copolymers, each copolymer comprising a chain length and composition; analyzing, by the device, the set of copolymers, and determining a set of features of the set of copolymers, the determined set of features at least corresponding to a chain length and composition of monomers; performing, by the device, a copolymer synthesis for each of the set of copolymers based on the determined set of features; determining, by the device, a copolymer complexation based on the performed copolymer synthesis; determining, by the device, a thermostability of a protein based on the determined copolymer complexation; executing, by the device, a regression model, the execution of the regression model being based on the thermostability of the protein; executing, by the device, an optimization model, the optimization model generating a copolymer design, the copolymer design comprising at least a chain length and composition of monomers of a copolymer that enables the thermostability of the protein; and applying, by the device, a selected copolymer to the protein, wherein the selected copolymer comprises a chain length and composition of monomers corresponding to the copolymer design.

In some embodiments, one or more systems and/or methods are disclosed which further include where the thermostability corresponds to the protein being stabilized to retain an activity at a predefined temperature over a predefined time interval, where the predefined temperature may be 37 degrees Celsius; and where the predefined time interval may be between 0-n hours (e.g., where n may be 168 hours, for example), where the copolymer design further corresponds to a determined retained enzyme activity (REA) for the predefined temperature over the predefined time interval. In some embodiments, by way of a non-limiting example, the predefined time interval may be 24 hours, as discussed herein.

In some embodiments, one or more systems and/or methods are disclosed which further include where the copolymer synthesis may be performed via the device executing an automated PET-RAFT, where the regression model may be a Gaussian regression model, where the optimization model includes an Bayesian optimization.

In some embodiments, one or more systems and/or methods are disclosed which further include where the protein is Chondroitinase ABC (ChABC), where the composition of monomers that causes ChABC to have a highest activity may be 23.1 mol % of 2-Diethylamino ethyl methacrylate (DEAEMA), 33.2 mol % of BMA, 31.7 mol % of Poly(ethyleneglycol) (n) monomethyl ether monomethacrylate (PEGMA), and 12.0 mol % of [2-(Methacryloyloxy)ethyl]trimethylammonium chloride solution (TMAEMA).

In some embodiments, one or more systems and/or methods are disclosed which further include where the monomers are selected from the group consisting of: 2-Diethylamino ethyl methacrylate (DEAEMA), Hydroxypropyl methacrylate (2-HPMA), [2-(Methacryloyloxy)ethyl]trimethylammonium chloride solution (TMAEMA), N-[3-(Dimethylamino)propyl]methacrylamide (DMAPMA), Methyl methacrylate (MMA), 3-Sulfopropyl methacrylate potassium salt (SPMA), Butyl methacrylate (BMA), and Poly(ethyleneglycol) (n) monomethyl ether monomethacrylate (PEGMA). In some embodiments, other suitable monomers may include, but are not limited to, the chemical structures depicted and provided in FIG. 3B.

### BRIEF DESCRIPTION OF THE DRAWINGS

The features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 is a block diagram of an example configuration within which the systems and methods disclosed herein could be implemented according to some embodiments of the present disclosure;

FIG. 2 is a block diagram illustrating components of an exemplary system according to some embodiments of the present disclosure;

FIG. 3A illustrates an exemplary composition configuration according to some embodiments of the present disclosure;

FIG. 3B illustrates exemplary chemical structures of monomers according to some embodiments of the present disclosure;

FIG. 4 illustrates an exemplary work flow according to some embodiments of the present disclosure;

FIG. 5 illustrates an exemplary work flow schematic according to some embodiments of the present disclosure;

FIGS. 6A-6C depict distributions of retained enzyme activity (REA) according to some embodiments of the present disclosure;

FIGS. 7A-7D depict further distributions of REA according to some embodiments of the present disclosure;

FIG. 8 depicts exemplary dynamic light scattering according to some embodiments of the present disclosure;

FIGS. 9A-9B depict exemplary cytotoxicity and information according to some embodiments of the present disclosure;

FIGS. 10-12 depict exemplary Tables S1-S3, respectively, for REA and compositional information according to some embodiments of the present disclosure; and

FIG. 13 is a block diagram illustrating an exemplary computing device used in various embodiments of the present disclosure.

### DETAILED DESCRIPTION

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

The present disclosure is described below with reference to block diagrams and operational illustrations of methods and devices. It is understood that each block of the block diagrams or operational illustrations, and combinations of blocks in the block diagrams or operational illustrations, may be implemented by means of analog or digital hardware and computer program instructions. These computer program instructions may be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks may occur out of the order noted in the operational illustrations. For example, two blocks shown in succession may in fact be executed substantially concurrently or the blocks may sometimes be executed in the reverse order, depending upon the functionality/acts involved.

For the purposes of this disclosure the term “server” should be understood to refer to a service point which provides processing, database, and communication facilities. By way of example, and not limitation, the term “server” may refer to a single, physical processor with associated communications and data storage and database facilities, or it may refer to a networked or clustered complex of processors and associated network and storage devices, as well as operating software and one or more database systems and application software that support the services provided by the server. Cloud servers are examples.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router (WR) mesh, or 2nd, 3rd, 4^{th} or 5^{th} generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or consumer or user) device, referred to as user equipment (UE)), may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device an Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

A client device (UE) may vary in terms of capabilities or features. Claimed subject matter is intended to cover a wide range of potential variations, such as a web-enabled client device or previously mentioned devices may include, but is not limited to, mass storage, one or more accelerometers, one or more gyroscopes, location-identifying type capability, or a display with a high degree of functionality, such as a touch-sensitive color 2D or 3D display, for example.

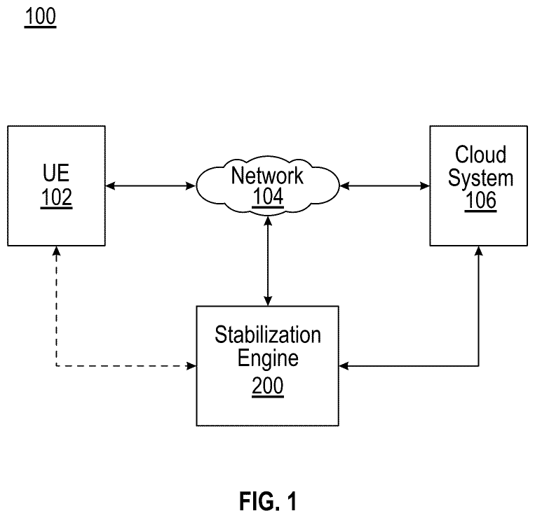

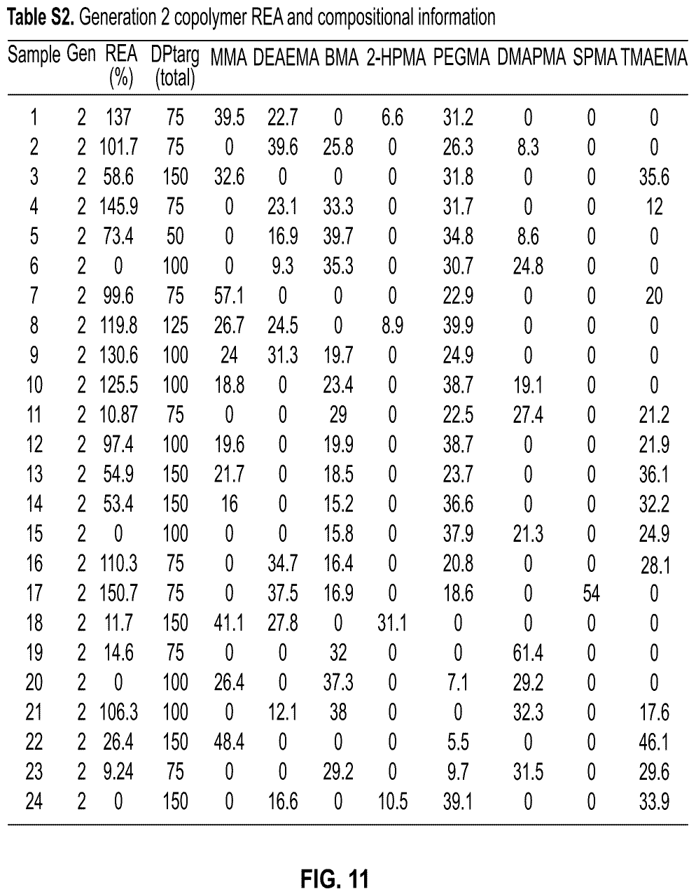

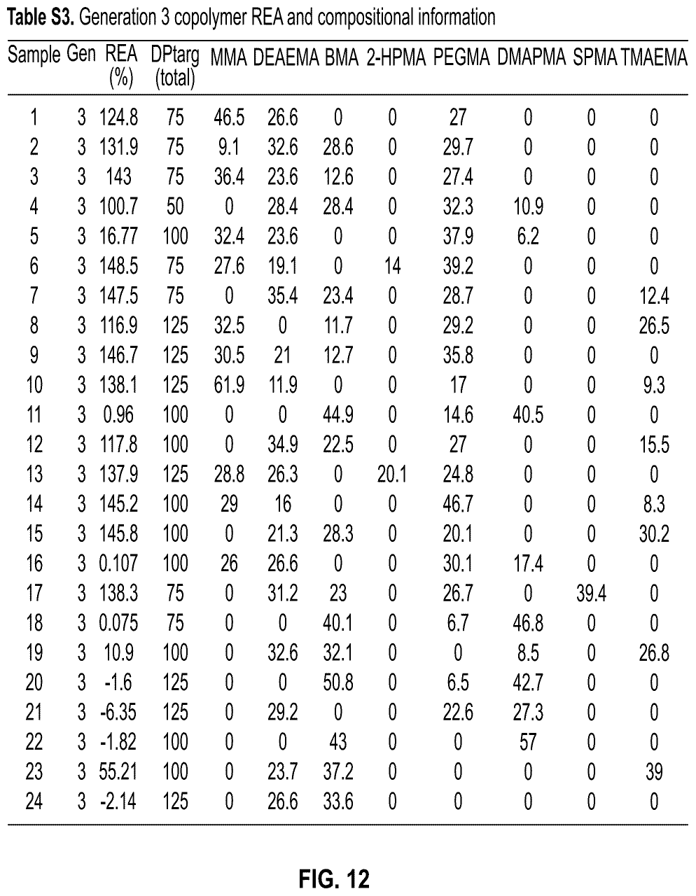

Certain embodiments and principles of the instant disclosure will now be described in greater detail. According to some embodiments, in connection with the subject matter disclosed and depicted in FIGS. 1-13, the disclosed systems and methods provide a novel framework for machine-assisted discovery of ChABC complexes towards sustained neural regeneration.

By way of background, Central Nervous System (CNS) injuries have devastating, long-term physical, psychological, and socio-economic consequences for patients and families. Although mortality rates have substantially improved in patients with CNS injuries, improvement in functional outcomes remains elusive. When it comes to functional recovery, many studies point to a general common theme: CNS neurons attempt to regenerate after a traumatic injury, but the post-injury environment is highly inhibitory and results in abortive regeneration. This is mainly due to the complex pathophysiology of the CNS, which undergoes enormous biochemical and physical changes post-injury.

Initial trauma results in extensive tissue damage, compromise of the blood-brain/spinal cord vasculature, and necrotic cell death. Compromised vasculature allows an influx of inflammatory cells that begin secreting pro-inflammatory cytokines and vasoactive peptides that potentiate further damage resulting in edema, excitotoxicity, altered gene expression, and enhanced cell signaling. Reactive astrocytes surround the site of injury and secrete a wide range of proinflammatory factors including chondroitin sulfate proteoglycans (CSPGs) resulting in a dense network of scar tissue that acts as a mechanical and chemical barrier to tissue regeneration.

Although glial scar and CSPGs within the scar play an important role in isolating injury site from healthy tissue, thereby minimizing uncontrolled tissue damage, their highly inhibitory nature sacrifices long-distance functional regeneration. CSPGs are known to be potent inhibitors of neurite outgrowth, and their inhibitory nature has been well documented in vitro and in vivo.

CSPG family members share two common features: a protein core that varies in structure, and glycosaminoglycan (GAG) side chains that vary in number and sulfation pattern. Extensive research has shown that high levels of CSPGs in CNS injury models in vivo correlates well with presence of dystrophic growth cones that are hallmarks of failed attempts at neuronal regeneration. The growth suppressive nature of CSPGs may be mainly attributed to their GAG side chains and sulfation pattern and degrading these inhibitory molecules has become a viable therapeutic strategy for promoting tissue regeneration.

As discussed herein, in some embodiments, Chondroitinase ABC (ChABC), which is a 115 kDa bacterial lyase that degrades the GAG side chains of CSPGs, has proven highly effective in promoting axonal sprouting and neuronal regeneration in various animal models. However, its use as a therapeutic may be limited by its thermal instability as it completely loses activity within a few hours at 37° C. under dilute conditions, as discussed above. In order to maintain therapeutic efficacy, continuous intrathecal administration every two weeks for up to six weeks may be required. Therefore, there may be an immediate need to develop highly stable, nanodispersed ChABC for glial scar degradation. Several approaches have been utilized for stabilizing ChABC, such as stabilization in high-concentration sugar solutions (1M trehalose, 2.5M sucrose), immobilization onto porous scaffolds, and protein structure modification. However, these approaches are ineffective, in that they do not provide the glial scare degradation, as discussed herein.

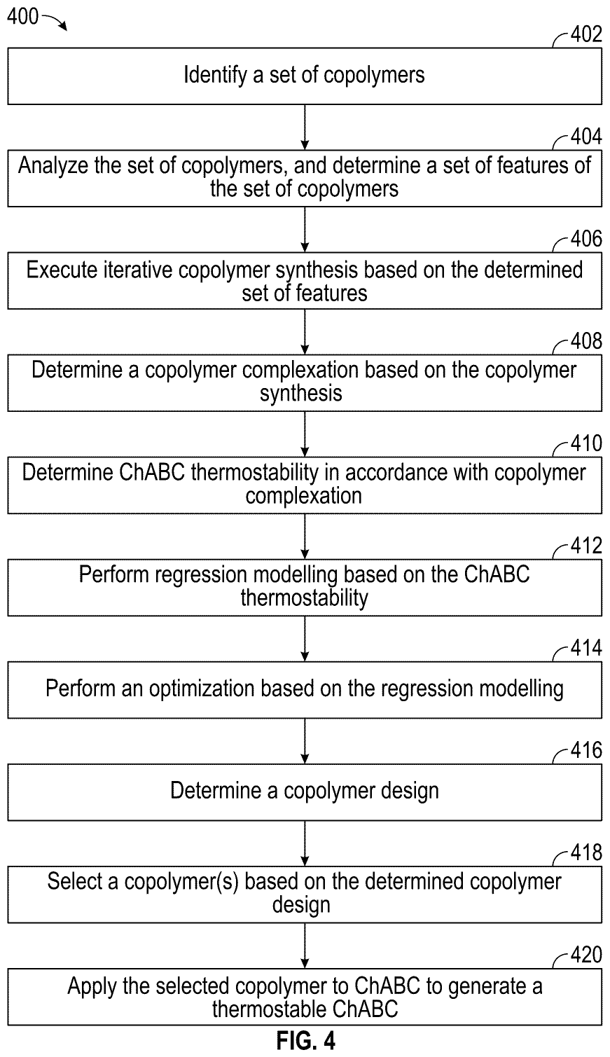

According to some embodiments, disclosed is a new polymer-enzyme complexes (PECs) of thermostabilized ChABC enabled by robotic synthesis, high-throughput testing, and data-driven optimization. Further discussion and detail of this processing is discussed in more detail below in relation to at least FIG. 4.

Accordingly, in some embodiments, PECs contain enzyme wrapped inside a synthetic chaperone-like polymeric shell that safeguards it from surrounding harsh microenvironments. However, rational identification of complementary copolymer composition on a case-by-case basis is highly time consuming and labor intensive. Meanwhile, data-driven design of copolymers has been pursued in silico. Recent advances in the field of oxygen tolerant polymer chemistries now enable synthesis of complex polymers in bench top well plates.

In some embodiments, to increase throughput, liquid-handling robotics may be programmed to perform autonomous polymer chemistry and post-polymerization functionalization in well plates, which increases synthetic efficiency while maintaining excellent reproducibility. This, among other benefits, may allow for automated and facile generation of datasets containing polymer chemistry and retained enzyme activity (REA) that may be effectively used to train machine learning (ML) models by a processor of a computer to represent the complex structure-activity relationship between polymer-enzyme interactions and REA. In some embodiments, using an active learning approach, three generations of 24 copolymer candidates may be proposed for synthesis and/or testing. From these 72 new copolymers, the most promising may be selected to conduct preliminary long-term studies, and the best of these may be selected for further biological and biophysical characterization.

With reference to FIG. 1, system 100 is depicted which includes UE 102 (e.g., a client device, as mentioned above), network 104, cloud system 106 and stabilization engine 200. UE 102 may be any type of device or instrument, such as, but not limited to, a mobile phone, wearable device, medical equipment, tablet, laptop, sensor, Internet of Things (IoT) device, autonomous machine, and any other device equipped with a cellular or wireless or wired transceiver.

In some embodiments, network 104 may be any type of network, such as, but not limited to, a wireless network, cellular network, the Internet, and the like (as discussed above). Network 104 facilitates connectivity of the components of system 100, as illustrated in FIG. 1.

Cloud system 106 may be any type of cloud operating platform and/or network based system (e.g. a medical platform or instrument platform, for example) upon which applications, operations, and/or other forms of network resources may be located. For example, system 106 may be a service provider, network provider and/or medical provider from where services and/or applications may be accessed, sourced or executed from. In some embodiments, cloud system 106 may include a server(s) and/or a database of information which is accessible over network 104. In some embodiments, a database (not shown) of cloud system 106 may store a dataset of data and metadata associated with local and/or network information related to a user(s) of UE 102 and the UE 102, and the services and applications provided by cloud system 106 and/or stabilization engine 200.

By way of a non-limiting example, cloud system 106 may be related to a medical facility or provider, whereby engine 200, discussed infra, provides functionality for performing ChABC stability, inter alia. Stabilization engine 200, as discussed above and below in more detail, may include components for providing functionality for machine-assisted discovery of ChABC complexes towards sustained neural regeneration. According to some embodiments, stabilization engine 200 may be a special purpose machine or processor and could be hosted by a device on network 104, within cloud system 106 and/or on UE 102. In some embodiments, engine 200 may be hosted by a server and/or set of servers associated with cloud system 106.

According to some embodiments, as discussed in more detail below, stabilization engine 200 may be configured to control a plurality of microservices, where each of the plurality of microservices are configured to execute a plurality of workflows associated with the discovery of ChABC complexes towards sustained neural regeneration.

According to some embodiments, as discussed above, stabilization engine 200 may function as an application provided by cloud system 106. In some embodiments, engine 200 may function as an application installed on a server(s), network location and/or other type of network resource associated with system 106. In some embodiments, engine 200 may function as application installed and/or executing on UE 102. In some embodiments, such application may be a web-based application accessed by UE 102 over network 104 from cloud system 106 (e.g., as indicated by the connection between network 104 and engine 200, and/or the dashed line between UE 102 and engine 200 in FIG. 1). In some embodiments, engine 200 may be configured and/or installed as an augmenting script, program or application (e.g., a plug-in or extension) to another application or program provided by cloud system 106 and/or executing on UE 102.

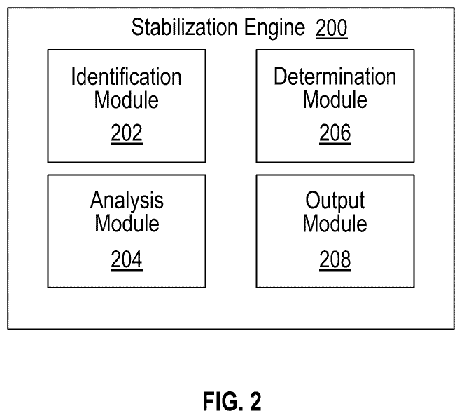

As illustrated in FIG. 2, according to some embodiments, stabilization engine 200 includes identification module 202; analysis module 204, determination module 208 and output module 210. It should be understood that the engine(s) and modules discussed herein are non-exhaustive, as additional or fewer engines and/or modules (or sub-modules) may be applicable to the embodiments of the systems and methods discussed. More detail of the operations, configurations and functionalities of engine 200 and each of its modules, and their role within embodiments of the present disclosure will be discussed below.

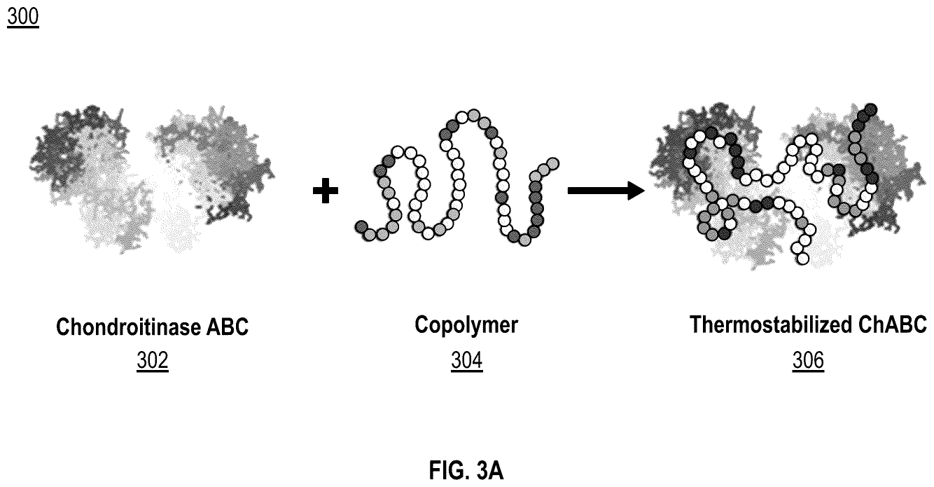

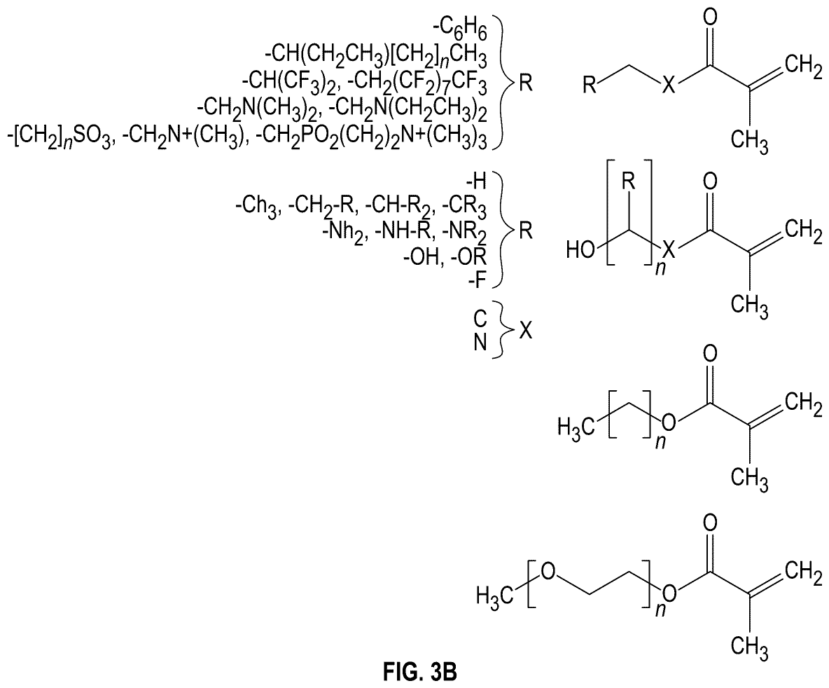

Turning to FIG. 3A, example 300, as discussed above, provides an exemplary configuration of a ChABC polymer enzyme complex that may be utilized sustained neural regeneration, as discussed herein.

As discussed above, ChABC 302 is a derived enzyme that has shown potential for treating spinal cord injuries because of its ability to degrade certain molecules in scar tissue and promote axonal regeneration. However, it may be highly unstable and may lose all of its activity within few hours at 37° C., which may necessitate the use of repeated injections or multiple infusions for days to weeks during medical treatment for treating the CNS.

In some embodiments, a copolymer 304 may be a polymer derived from more than one species of monomer. The polymerization of monomers into copolymers is called copolymerization. Copolymers obtained by copolymerization of two monomer species are sometimes called bipolymers. Those obtained from three and four monomers are called terpolymers and quaterpolymers, respectively.

According to some embodiments, other suitable monomers may include, but are not limited to, the chemical structures depicted and provided in FIG. 3B, as discussed above.

Commercial copolymers include acrylonitrile butadiene styrene (ABS), styrene/butadiene co-polymer (SBR), nitrile rubber, styrene-acrylonitrile, styrene-isoprene-styrene (SIS) and ethylene-vinyl acetate, all formed by chain-growth polymerization. Another production mechanism may be step-growth polymerization, used to produce the nylon-12/6/66 copolymer[2] of nylon 12, nylon 6 and nylon 66, as well as the copolyester family.

Since a copolymer may typically consist of at least two types of constituent units (also structural units), copolymers may be classified based on how these units are arranged along the chain. [3] Linear copolymers consist of a single main chain, and include alternating copolymers, statistical copolymers and block copolymers. Branched copolymers consist of a single main chain with one or more polymeric side chains, and may be grafted, star shaped or have other architectures.

Thus, as illustrated in example 300 depicted in FIG. 3A, as discussed herein, a thermostabilized ChABC 306 may be produced via a combination of ChABC 302 and a copolymer 304. Thermostabilized ChABC 306 may evidence increased regrowth, sprouting and functionality recovery after treatment of the CNS (e.g., SCI or TBI, for example), as discussed herein.

Turning to FIG. 4, Process 400 is disclosed which provides non-limiting example embodiments for the neural regeneration via stabilized ChABC, as discussed herein.

As discussed above, among the many molecules that contribute to glial scarring, chondroitin sulfate proteoglycans (CSPGs) are known to be potent inhibitors of neuronal regeneration. ChABC, which is a bacterial lyase, is known to degrade the glycosaminoglycan (GAG) side chains of CSPGs and promote tissue regeneration. However, ChABC may be thermally unstable and loses all activity within a few hours at 37° C. under dilute conditions.

As discussed herein, in at least some embodiments, to address these shortcomings, disclosed is Process 400 which provides a framework for machine-assisted discovery of ChABC complexes towards sustained neural regeneration. In some embodiments, the framework may leverage a determination of a diverse set of tailor-made random copolymers that complex and stabilize ChABC at physiological temperature. The copolymer designs, which are based on chain length and/or composition of the copolymers, may be identified using an active machine learning paradigm, which involves, but is not limited to, copolymer synthesis, testing for ChABC thermostability upon copolymer complexation, Gaussian Process Regression modeling and Bayesian optimization. Copolymers may be synthesized by automated PET-RAFT, and thermostability of ChABC may be assessed by retained enzyme activity (REA) after a predefined number of hours at 37° C. In some embodiments, the predefined number of hours may be 24 hours. In some embodiments, the hours may correspond to a range of hours, for example, between 0-168 hours.

In some embodiments, with regard to automated PET-RAFT, polymer libraries may be prepared. For example, in some embodiments, Hamilton MLSTARlet sequences and processes may be generated from Python with sample concentration, reagent volumes, and well position. Files containing reaction information may be transferred to the Hamilton MLSTARlet to prime the robotic transfers. Stock solutions of monomer (2 M), ethyl 2-(phenylcarbonothioylthio)-2-phenylacetate (RAFT agent, 100 or 50 mM) and ZnTPP (4 or 2 mM) may be prepared in DMSO and pipetted into 1 mL aliquots. Aliquots may be loaded into a Hamilton MLSTARlet liquid handling robot and automatically pipetted into 96-well clear flat-bottom polypropylene well plates (Greiner bio-one). In at least some embodiments, monomer/CTA ratio may be varied from 10-1000 while ZnTPP/CTA remained at 0.01. Polymer mixtures may be dispensed to a total volume of 200 μL and a final monomer concentration of 1 M. They may then be covered with a well-plate sealing tape and radiated under 560 nm LED light (5 mW/cm2, TCP 12-Watt Yellow LED BR30 bulb) for 0-72 h.

In some embodiments, implementation of the disclosed systems and methods (as provided via Process 400) demonstrates significant improvements in REA via implementations of active learning while identifying exceptionally high-performing copolymers. For example, one designed copolymer may promote residual ChABC activity, for example, without limitation, near 30%, even after one week, and notably outperformed other common stabilization methods for ChABC. Together, these results along with the instant disclosure's provided techniques highlight a novel pathway towards sustained tissue regeneration.

According to some embodiments, Step 402 of Process 400 may be performed by identification module 202 of stabilization engine 200; Steps 404-406 and 412-414 may be performed by analysis module 204; Steps 408-410 and 416-418 may be performed by determination module 206; and Step 420 may be performed by output module 208.

Process 400 begins with Step 402 where engine 200 may identify a set of copolymers. According to some embodiments, engine 200 may utilize any type of known or to be known machine learning (ML) and/or artificial intelligence (AI) algorithm or classifier to identify polymer designs with a high likelihood of stabilizing ChABC. By way of non-limiting example, such algorithms and/or classifiers may involve, but are not limited to, computer vision, neural networks, regression modelling (e.g., logistic regression, for example), Bayesian optimization, feature vector analysis, data mining, and the like, or some combination thereof. In some embodiments, the identified set of copolymers may be selected via a search of a database or library of copolymers.

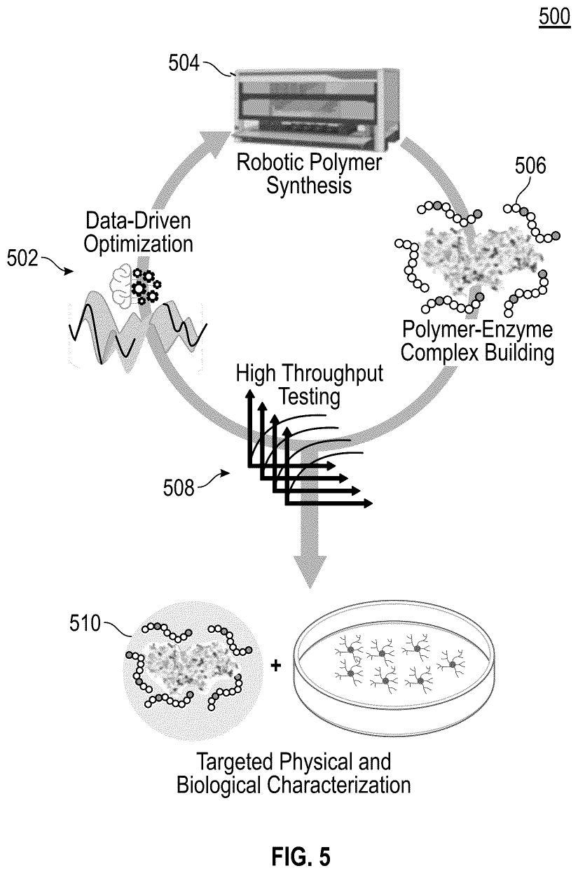

According to some embodiments, by way of example as depicted in FIG. 5, optimization 500 of an ML/AI model may involve robotic data-driven optimization 502, robotic polymer synthesis 504, polymer-enzyme complex building 506 and high-throughput testing 508, which may result in targeted physical and biological characterization 510. Such processing of optimization 500 may be tied to the processing steps of Process 400, as evidenced from the discussion herein, and discussed above in relation to the discovery of new PECs.

According to some embodiments, copolymers may be restricted to have four or fewer distinct monomers (e.g., methacrylates or a methacrylamide), and chain lengths with degree of polymerization (DP) between 10 and 1000.

Therefore, the active learning process embodied in optimization 500, via Step 402, may evidence a data set seeded with, for example, 504 copolymers with systematic variation in monomer composition and chain length and accompanying data on their ability to stabilize ChABC, as quantified by REA at 37° C. for according to a predefined interval of hours (e.g., 24 hours or between 0-168 hours). Such identification may be performed via ML/AI processing of a library of copolymers, and/or a search for copolymers with such configuration.

In some embodiments, selected polymers may have a molecular weight (M_{w} and Mₙ) and dispersity (Ð) that may be measured by gel permeation chromatography (GPC) using an Agilent 1260 Infinity II. In some embodiments, polymer samples may be eluted through a Phenomenex 5.0 μm guard column (50×7.5 mm) preceded by two Phenomenex Phenogel columns (10E4 and 10E3 Å). In some embodiments, GPC calibration may be completed with Agilent PMMA standards. According to some embodiments, polymers may be identified at 50:1 eluent/polymer ratio in DMF and filtered with a 0.45 μm PTFE filter. In some embodiments, polymer conversion may be calculated by obtaining 1H NMR spectra using a Varian VNMRS 500 MHz spectrometer with mesitylene as an internal standard and processed using Mestrenova 11.0.4.

In Step 404, engine 200 may analyze the set of copolymers, and determine a set of features of the set of copolymers. According to some embodiments, such analysis may involve utilizing any type of known or to be known ML/AI algorithm or technique, as discussed above in relation to Step 402. According to some embodiments, the employed ML/AI algorithms and techniques may include any type of known or to be known classifier and/or computational analysis technique that may be utilized on a set of copolymers and/or its associated and/or inherent data, as discussed below.

As a result, the features of the copolymers may be identified, which may indicate, but are not limited to, a number of copolymers, a chain length information, composition information, monomer information, temperature for REA, time for REA, and the like, or some combination thereof.

According to some embodiments, techniques executed by engine 200 may involve the Python libraries Scikit-learn v0.24.1 and Hyperopt v0.2.5 used to optimize Gaussian Process Regression (GPR) models to predict REA from a feature vector describing a copolymer. For example, in some embodiments, an input feature vector for each copolymer may be nine-dimensional with the chain length divided by 200 (the maximum prescribed chain length) in the first dimension and the fractional incorporation of each of the eight possible monomers in the remaining dimensions. According to some embodiments, nested k-fold cross-validation may be used to construct the GPR models prior to each generation of proposed polymers. In some embodiments, for example, the dataset of copolymers may be first split into five outer folds. For each outer fold, a set of optimal hyperparameters (e.g., those present in the radial basis function kernel and white noise kernel) may be obtained by 20-fold cross-validation over the remaining outer folds. This may result in five sets of hyperparameters, which may be averaged to obtain a final set of hyperparameters.

In some embodiments, using such hyperparameters, a final GPR model may be trained with all experimental data acquired to that point. According to some embodiments, models may be optimized by minimizing the average mean squared error of a power-transform of the REA. The final GPR model may be used in tandem with a Bayesian optimization scheme to identify 200 new copolymers that maximize an expected improvement (EI) acquisition function.

According to some embodiments, engine 200 may use an EI function that contains a parameter controlling the balance between exploration and exploitation, where, in some embodiments, each of the 200 candidates may be generated with a unique value of this parameter.

According to some embodiments, candidates may be downselected to 24 polymers using an unsupervised learning approach based on the DBSCAN and k-Means clustering algorithms, which may be performed to promote diversity in the polymers proposed for synthesis (e.g., a randomness at a threshold level). Following experimental synthesis and evaluation of the 24 proposed polymers, this newly acquired data may be added to the dataset, and the process, beginning with the training of a new GPR model may be repeated a predetermined number of times (e.g., 3, for example). In some embodiments, a first generation of 24 polymer candidates was produced employing a GPR trained on a human-crafted dataset of 504 polymer/REA combinations.

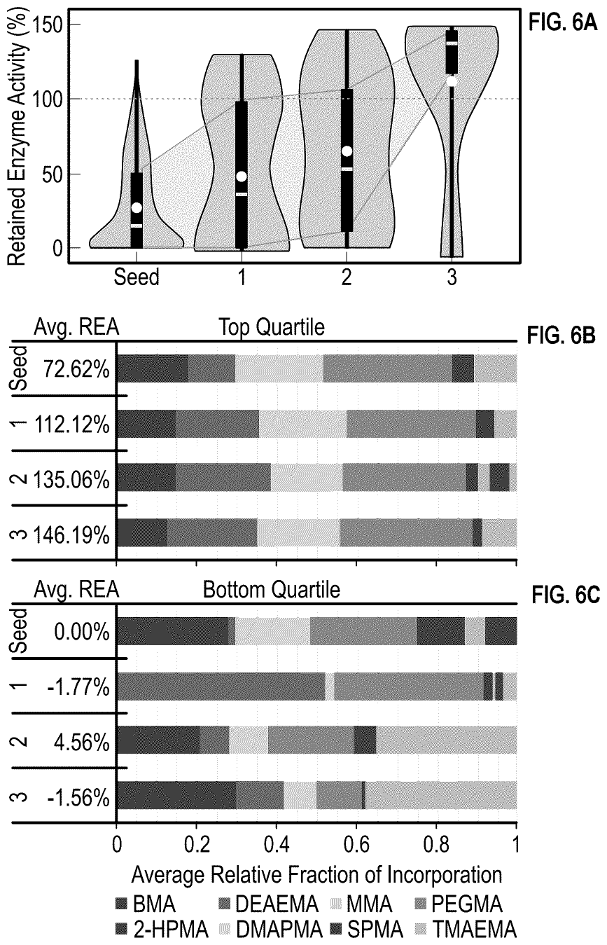

Turning to FIGS. 6A-6C, illustrated are comparisons of distributions of REA for ChABC-PECs for the seed database and implementations of active learning. In FIG. 6A, depicted is a distribution of REAs of all samples tested, including the seed database and each generation of candidates produced by the active learning scheme. As depicted implementations (1-3) of active learning result in remarkable improvement of REA relative to the seed data, with many designs yielding enhanced activity above 100%. This may be reflected by an overall upward trend of the distribution of data in terms of quartile positions, median, mean, and maximum. In the original seed dataset, an average REA of 27.1% for 504 samples was observed, with one sample exhibiting 125% of the native enzyme activity at the end of 24 hours. In some embodiments, an average REA of samples across different generations improved with global average REAs of 50.3, 64.2, and 111.4% for generations 1, 2, and 3, respectively. In some embodiments, a highest performing samples for generations 1, 2, and 3 exhibited REAs of 129.4, 146, and 148%, respectively.

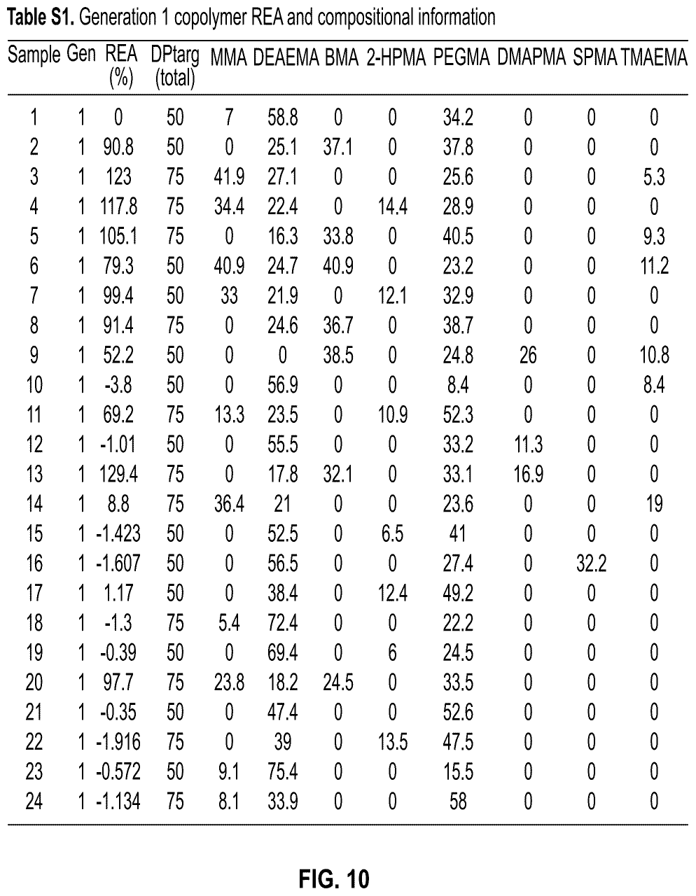

According to some embodiments, details of the copolymers along with REA for generations 1,2 and 3 are depicted in Tables S1-S3—FIGS. 10-12, respectively.

FIG. 6B presents an aggregated analysis of the average REA and average polymer composition for ChABC-PECs in the top quartile for REA. FIG. 6C depicts shows an analysis for those in the bottom quartile.

According to some embodiments, variations of average composition of the top quartiles of the different sets of polymers are small, especially when compared to those of the bottom quartiles. Nevertheless, the average REA increases upon successive implementation of active learning. This indicates a delicate balance of polymer chemistry may be required to achieve high-performing polymers and highlights the underlying challenge of the polymer design task.

According to some embodiments, comparing the second and third generations, the complete removal of DMAPMA and SPMA increases average REA by more than 10%. This may be likely related to a notable imbalance between the fraction of more hydrophobic vs. more hydrophilic monomers. In some embodiments, the active learning process may identify that a preponderance of hydrophilic monomers may be more likely associated with high REA. The depicted observations of FIGS. 6A-6C highlight the importance of implementing an active learning approach, since it may capture fine details that could easily escape a rational design approach, and it has a higher probability of success compared to a random or systematic search.

Continuing with Process 400, engine 200 may then execute Step 406 where a copolymer synthesis may be executed. In some embodiments, the input of the copolymer synthesis may include, but is not limited to, the determined set of features of the copolymers. According to some embodiments, such syntheses may be performed via the ML/AI algorithms discussed above, and may involve the first, second and third generations as discussed above at least in relation to FIGS. 6A-6C. In some embodiments, engine 200 may execute automated PET-RAFT to perform the copolymer synthesis, as discussed above.

In Step 408, engine 200 may then determine a copolymer complexation based on the copolymer synthesis. According to some embodiments, such syntheses may be performed via the ML/AI algorithms discussed above, and as discussed in relation to FIGS. 5-6C.

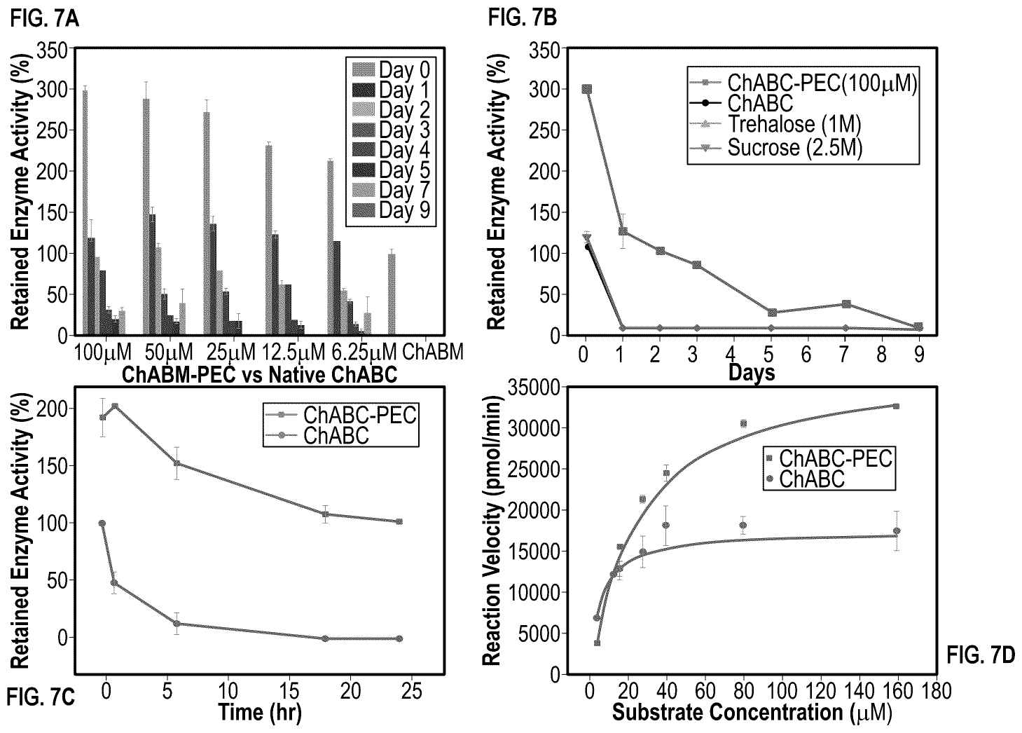

According to some embodiments, as depicted in FIGS. 7A-7D, REA for analyzed complexations are depicted. According to some embodiments, FIG. 7A depicts a REA of ChABC in the presence of polymer at different concentrations at 37° C. For example, in some embodiments, PECs at different concentrations increased activity of enzyme at t=0 at least 2-3 fold and maintained high levels of enzyme for the initial few days. FIG. 7B provides a comparison of PEC with common enzyme stabilizers Trehalose and Sucrose. FIG. 7C provides PEC retained >100% activity while native ChABC lost all activity within 24 hours. And, FIG. 7D provides activity of native ChABC and ChABC-PEC (12.5 μM) at varying substrate concentrations.

According to some embodiments, long-term stability of the best PEC may be identified during active learning in aCSF as well as the effect of copolymer concentration. For example, in some embodiments, a candidate that had the highest REA retention of ChABC after 24 hours may be chosen. In some embodiments, a composition of the best performing copolymer may be DEAMA (0-100 mol %): BMA (0-100 mol %): PEGMA (0-100 mol %): TMAEMA (0-100 mol %) and had a DP of 10-1000. In some embodiments, the above ranges may be +/−5 mol % with the restriction that the sum of all mol % be 100%. By way of a non-limiting example, a composition of the best performing copolymer may be DEAMA (23.1 mol %): BMA (33.2 mol %): PEGMA (31.7 mol %): TMAEMA (12.0 mol %) and had a DP of 75; the molecular weight of the copolymer is 33 kDa and has a dispersity of Ð=1.5.

In some embodiments, for example, five copolymer concentrations may be tested for a long-term stability assessment to determine an effect of copolymer concentration on enzyme activity retention. Enzyme concentration may be maintained at 0.1-10,000 ng/μL (for example, e.g., 2 ng/μL (17.63 nM)) for long-term stability experiments and copolymer concentrations may be varied from 1-1000 M. According to some embodiments, copolymer at all concentrations may be evidenced to increase the initial activity of the enzyme at t=0. For example, the initial activity of the enzyme increased by 3-fold for 100 and 50 μM copolymer concentrations while 2-2.5-fold increases were observed for 25, 12.5, and 6.25 μM concentrations (as depicted in FIG. 7A).

In some embodiments, for example, at the end of 24 hours, PECs at all five concentrations retained around 110-150% native enzyme activity within the same period. The native enzyme did not retain any activity at the end of 24 hours. By contrast, ChABC-PECs continued to retain high levels of activity (>60%) at the end of 72 hours. By the end of day 7 (168 hours), samples at 25 and 12.5 μM lost all activity but 100, 50, and 6.25 μM samples continued to have REAs of 29%, 39%, and 28%, respectively.

In some embodiments, an efficiency of PECs to stabilize ChABC may be compared with common enzyme stabilizers trehalose and sucrose at an enzyme concentration of 2 ng/μL (17.63 nM). In some embodiments, based on such efficiency comparison, ChABC stabilized with 1M trehalose and 2.5 M sucrose lost all enzyme activity within 24 hours while PEC retained 100% activity within the same time period (which is depicted in FIGS. 7B and 7C).

In some embodiments, kinetic parameters of ChABC in the presence of the set of copolymers may realize an increase in the maximum velocity (Vₘₐₓ), while the affinity to the substrate decreased, as may be observed from Km (as depicted in FIG. 7D). Further disclosure of this embodiment may be found below in Table 1:

TABLE 1
Kinetic parameters of ChABC with and without copolymers:

| | Vₘₐₓ | | |
|---|---|---|---|
| Sample | (pmol/μg/min) | Kₘ (μM) | K_{cat} (s⁻¹) |
| ChABC | 18,070 | 5.01 | 284.7 |
| ChABC-PEC | 37,315 | 23.62 | 587.93 |

Accordingly, in some embodiments, as depicted in FIG. 7C, the lower activity of the native enzyme compared to ChABC-PEC may be because of substrate inhibition, which may be prevented in the presence of the copolymer resulting in nanodispersed PEC.

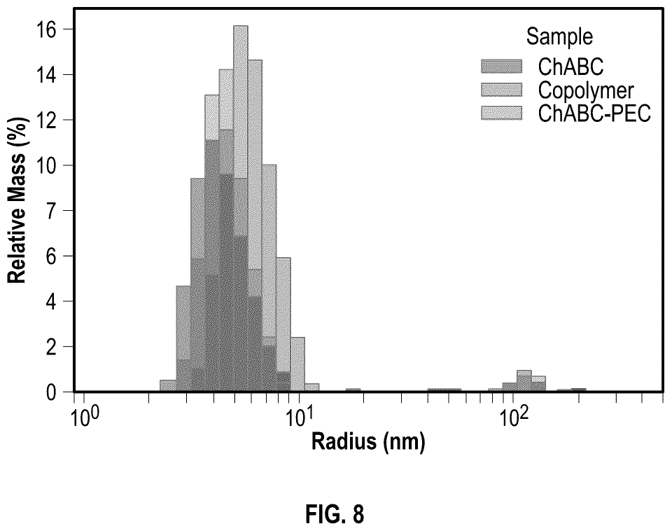

Continuing with Process 400, engine 200 may execute Step 410 and determine CHABC thermostability in accordance with the copolymer complexation. In some embodiments, to characterize the size of PECs in solution, the hydrodynamic size of copolymers, native enzyme, and PEC may be measured using dynamic light scattering (DLS). In some embodiments, as depicted in FIG. 8, scattering data suggest that the copolymer may be swollen in solution but complexes around nanodispersed ChABC without forming multi-molecular species.

According to some embodiments, DLS of polymers and polymer-enzyme mixtures may be performed on a DynaPro DLS Plate Reader III, Wyatt Technologies. Concentration of ChABC for DLS implementations may be maintained at 0.2 mgmL⁻¹ while polymer concentration may be at 2 mgmL⁻¹. As in Step 410, data may be collected using a wavelength of 830 nm and a scattering angle of 173°. A predetermined number of acquisitions may be collected for each sample (e.g., 15, for example) with an acquisition time of n seconds per acquisition (e.g., 5 seconds, for example) using auto attenuation. Regularization analysis may be performed using Rayleigh spheres model for hydrodynamic size measurement.

According to some embodiments, as discussed above, Step 410 may result in identification of copolymer designs, indicative of chain length and/or composition of the copolymers, which may, in connection with ChABC, result in thermostability of ChABC assessed by REA after a predefined interval of hours at 37° C. (e.g., where, in some embodiments, the predefined interval may correspond to a range of 0-168 hours, and in some embodiments, the predefined interval may correspond to 24 hours).

In Step 412, engine 200 may perform regression modelling based on the ChABC thermostability. In some embodiments, engine 200 may utilize a Gaussian Process Regression model(s), with the information determined from Step 410 as the input, to perform the disclosed regression modelling.

According to some embodiments, the determined information related to thermostability may be based on and/or involve a Thermal Stability Assay. In some embodiments, activity of PECs may be evaluated by their ability to digest chondroitin sulfate substrate resulting in unsaturated disaccharides. For example, polymers may be synthesized and diluted in DMSO before further dilution into assay buffer (for example, e.g., aCSF: 149 mM NaCl, 3 mM KCl, 0.8 mM MgCl₂, 1.4 mM CaCl₂ 1.5 mM Na₂HPO₄, 0.2 mM NaH₂PO₄, pH 7.4) to a final concentration of 25 μM (<1% DMSO).

In some embodiments, copolymers that either gelled in DMSO due to high hydrophobicity or precipitated in aCSF buffer may be excluded. All stabilizations may be performed for 0-168 hours at 37° C. and at a range of polymer concentrations 0-1000 μM. In some embodiments, for example, 15 μL of 0-1000 μM polymer samples may be mixed with 15 μL of 0.1-10,000 ng/μL ChABC (8.69 nM) in UV-star polystyrene 384 well plates that were thermally sealed with a plate sealing film before being thermally challenged in an incubator at 37° C. for 24 hours. Substrate solution may be prepared by diluting 10 mg/mL of chondroitin sulfate in DI water to a final concentration of 4 mg/mL in assay buffer. For example, in some embodiments, 30 μL of 4 mg/mL substrate was added to 30 μl of polymer-enzyme complexes and an increase in absorbance may be measured in kinetic mode for 60 mins at 232 nm with 20 sec intervals at 30° C.

According to some embodiments, an initial rate of change of absorbance may be used to calculate a specific enzyme activity using the following equation.

$$Specific Activity = \begin{matrix}\frac{Adjusted V max ( \frac{OD}{min} ) \times well volume ( L ) \times 1 {0}^{12} ( \frac{p mol}{mol} )}{ext coeff ( {M}^{- 1} \cdot {cm}^{- 1} ) \times path correction \times amount of enzyme ( μ g )} & ( 1 )\end{matrix}$$

Where, for example, ext Coeff for CS substrate was 3800 M⁻¹ cm⁻¹ and path correction may be 0.95 cm.

In Step 414, engine 200 may perform an optimization based on the regression modelling performed in Step 412. In some embodiments, engine 200 may execute, for example, a Bayesian optimization with the results from Step 412 as the input to such optimization model.

In Step 416, based on the optimization performed in Step 414, engine 200 may determine a copolymer design. The determined copolymer design may include, but is not limited to, a chain length, composition type, number and/or type of monomer, determined temperature for REA, determined time for REA, and the like, or some combination thereof, which enables thermostability for ChABC. In some embodiments, such design may be stored in a database as a data structure.

In Step 418, engine 200 may then select a set of copolymers that adhere to the copolymer design; and in Step 420, apply the selected copolymer to the ChABC to generate a thermostable ChABC, as discussed above (and illustrated in FIG. 3A).

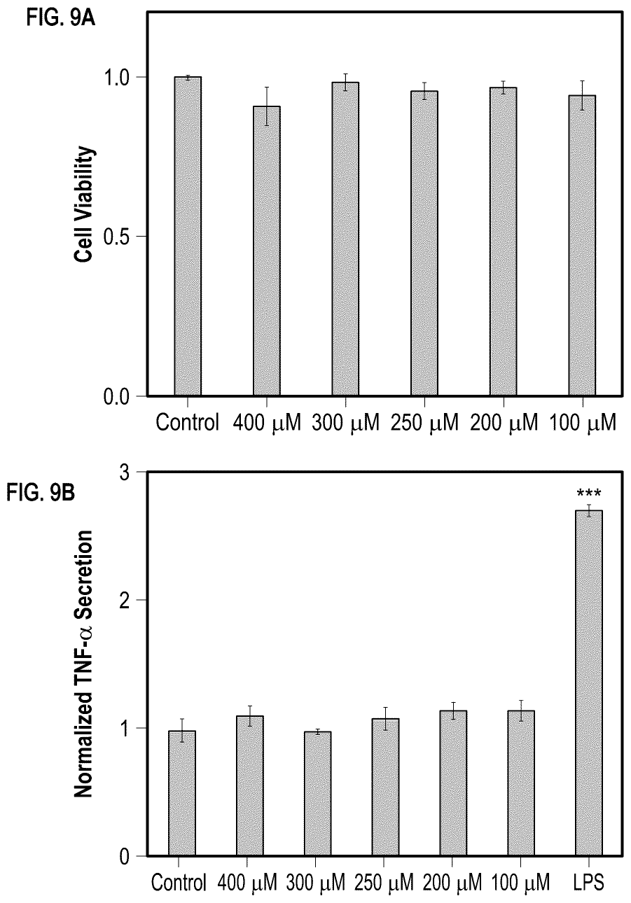

According to some embodiments, any possible adverse cytotoxic effects of copolymers may be treated via astrocytes. In some embodiments, for example, astrocytes may be seeded in 24 well plates and treated with different concentrations of copolymer (of the determine design from Steps 416-420). After 24 hours, supernatants may be collected, and cell viability may be assessed using standard Live/Dead assay. As depicted in FIG. 9A, cytotoxicity values are provided, whereby no cytotoxicity may be observed even at the highest concentration of 400 μM (7.2 mg/L). In some embodiments, a wide range of concentrations from 400 μM to 6.25 μM may be tested for possible cytotoxicity and no adverse effects may be observed at all concentrations (data shown until 100 μM).

FIG. 9B depicts an embodiment, for example, that involves the evaluation of inflammatory properties of the designed copolymers that could stimulate the secretion of Tumor Necrosis Factor-alpha (TNF-α) in astrocytes. Lipopolysaccharide (LPS) is a well-known inflammatory agent that upregulates the secretion of TNF-α in astrocytes and has been well characterized. Astrocytes treated with LPS had high levels of TNF-α secretion while astrocytes treated with copolymers had no significant differences compared to control group (no LPS).

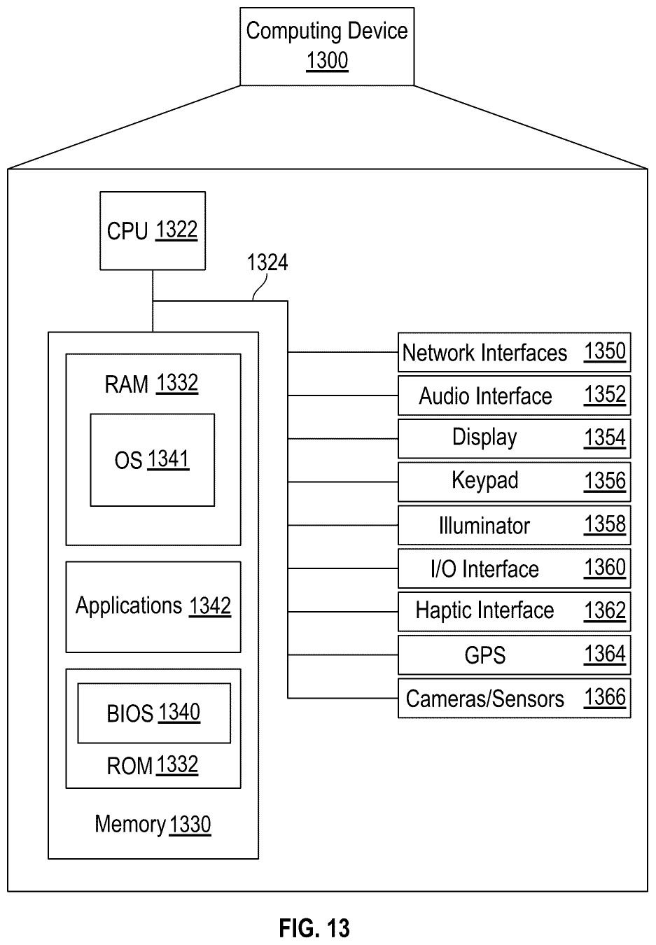

FIG. 13 is a block diagram illustrating a computing device 1300 showing an example of a client or server device used in the various embodiments of the disclosure. Computing device 1300 may be a representation of UE 102, as mentioned above.

The computing device 1300 may include more or fewer components than those shown in FIG. 13, depending on the deployment or usage of the device 1300. For example, a server computing device, such as a rack-mounted server, may not include audio interfaces 1352, displays 1354, keypads 1356, illuminators 1358, haptic interfaces 1362, GPS receivers 1364, or cameras/sensors 1366. Some devices may include additional components not shown, such as graphics processing unit (GPU) devices, cryptographic co-processors, artificial intelligence (AI) accelerators, or other peripheral devices.

As shown in FIG. 13, the device 1300 includes a central processing unit (CPU) 1322 in communication with a mass memory 1330 via a bus 1324. The computing device 1300 also includes one or more network interfaces 1350, an audio interface 1352, a display 1354, a keypad 1356, an illuminator 1358, an input/output interface 1360, a haptic interface 1362, an optional GPS receiver 1364 (and/or an interchangeable or additional GNSS receiver) and a camera(s) or other optical, thermal, or electromagnetic sensors 1366. Device 1300 may include one camera/sensor 1366 or a plurality of cameras/sensors 1366. The positioning of the camera(s)/sensor(s) 1366 on the device 1300 may change per device 1300 model, per device 1300 capabilities, and the like, or some combination thereof.

In some embodiments, the CPU 1322 may comprise a general-purpose CPU. The CPU 1322 may comprise a single-core or multiple-core CPU. The CPU 1322 may comprise a system-on-a-chip (SoC) or a similar embedded system. In some embodiments, a GPU may be used in place of, or in combination with, a CPU 1322. Mass memory 1330 may comprise a dynamic random-access memory (DRAM) device, a static random-access memory device (SRAM), or a Flash (e.g., NAND Flash) memory device. In some embodiments, mass memory 1330 may comprise a combination of such memory types. In one embodiment, the bus 1324 may comprise a Peripheral Component Interconnect Express (PCIe) bus. In some embodiments, the bus 1324 may comprise multiple busses instead of a single bus.

Mass memory 1330 illustrates another example of computer storage media for the storage of information such as computer-readable instructions, data structures, program modules, or other data. Mass memory 1330 stores a basic input/output system (“BIOS”) 1340 for controlling the low-level operation of the computing device 1300. The mass memory also stores an operating system 1341 for controlling the operation of the computing device 1300.

Applications 1342 may include computer-executable instructions which, when executed by the computing device 1300, perform any of the methods (or portions of the methods) described previously in the description of the preceding Figures. In some embodiments, the software or programs implementing the method embodiments may be read from a hard disk drive (not illustrated) and temporarily stored in RAM 1332 by CPU 1322. CPU 1322 may then read the software or data from RAM 1332, process them, and store them to RAM 1332 again.

The computing device 1300 may optionally communicate with a base station (not shown) or directly with another computing device. Network interface 1350 is sometimes known as a transceiver, transceiving device, or network interface card (NIC).

The audio interface 1352 produces and receives audio signals such as the sound of a human voice. For example, the audio interface 1352 may be coupled to a speaker and microphone (not shown) to enable telecommunication with others or generate an audio acknowledgment for some action. Display 1354 may be a liquid crystal display (LCD), gas plasma, light-emitting diode (LED), or any other type of display used with a computing device. Display 1354 may also include a touch-sensitive screen arranged to receive input from an object such as a digit from a human hand.

Keypad 1356 may include any input device arranged to receive input from a user. Illuminator 1358 may provide a status indication or provide light.

The computing device 1300 also includes an input/output interface 1360 for communicating with external devices, using communication technologies, such as USB, infrared, Bluetooth™M, or the like. The haptic interface 1362 provides tactile feedback to a user of the client device.

The optional GPS transceiver 1364 may determine the physical coordinates of the computing device 1300 on the surface of the Earth, which typically outputs a location as latitude and longitude values. GPS transceiver 1364 may also employ other geo-positioning mechanisms, including, but not limited to, triangulation, assisted GPS (AGPS), E-OTD, CI, SAI, ETA, BSS, or the like, to further determine the physical location of the computing device 1300 on the surface of the Earth. In one embodiment, however, the computing device 1300 may communicate through other components, provide other information that may be employed to determine a physical location of the device, including, for example, a MAC address, IP address, or the like.

It is understood that at least one aspect/functionality of various embodiments described herein may be performed in real-time and/or dynamically. As used herein, the term “real-time” is directed to an event/action that may occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation may be used in guiding the physical process.

As used herein, the term “dynamically” and term “automatically,” and their logical and/or linguistic relatives and/or derivatives, mean that certain events and/or actions may be triggered and/or occur without any human intervention. In some embodiments, events and/or actions in accordance with the present disclosure may be in real-time and/or based on a predetermined periodicity of at least one of: nanosecond, several nanoseconds, millisecond, several milliseconds, second, several seconds, minute, several minutes, hourly, several hours, daily, several days, weekly, monthly, and the like.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical, or other forms of propagated signals (e.g., carrier waves, infrared signals, digital signals, and the like), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, and the like).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores,” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, and the like).

In some embodiments, one or more of illustrative computer-based systems or platforms of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

In some embodiments, as detailed herein, one or more of the computer-based systems of the present disclosure may obtain, manipulate, transfer, store, transform, generate, and/or output any digital object and/or data unit (e.g., from inside and/or outside of a particular application) that may be in any suitable form such as, without limitation, a file, a contact, a task, an email, a message, a map, an entire application (e.g., a calculator), data points, and other suitable data. In some embodiments, as detailed herein, one or more of the computer-based systems of the present disclosure may be implemented across one or more of various computer platforms such as, but not limited to: (1) FreeBSD, NetBSD, OpenBSD; (2) Linux; (3) Microsoft Windows™; (4) Open VMS™; (5) OS X (MacOS™); (6) UNIX™; (7) Android; (8) iOS™; (9) Embedded Linux; (10) Tizen™; (11) WebOS™; (12) Adobe AIR™; (13) Binary Runtime Environment for Wireless (BREW™); (14) Cocoa™ (API); (15) Cocoa™ Touch; (16) Java™ Platforms; (17) JavaFX™; (18) QNX™; (19) Mono; (20) Google Blink; (21) Apple WebKit; (22) Mozilla Gecko™M; (23) Mozilla XUL; (24) .NET Framework; (25) Silverlight™; (26) Open Web Platform; (27) Oracle Database; (28) Qt™; (29) SAP NetWeaver™; (30) Smartface™; (31) Vexi™M; (32) Kubernetes™ and (33) Windows Runtime (WinRT™) or other suitable computer platforms or any combination thereof. In some embodiments, illustrative computer-based systems or platforms of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, illustrative computer-based systems or platforms of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In some embodiments, illustrative computer-based systems or platforms of the present disclosure may be configured to output to distinct, specifically programmed graphical user interface implementations of the present disclosure (e.g., a desktop, a web app., and the like). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, or objects. For example, such projections may be a visual overlay for a mobile augmented reality (MAR) application.

As used herein, the term “mobile device,” or the like, may refer to any portable electronic device that may or may not be enabled with location tracking functionality (e.g., MAC address, Internet Protocol (IP) address, or the like). For example, a mobile electronic device may include, but is not limited to, a mobile phone, Personal Digital Assistant (PDA), Blackberry™, Pager, Smartphone, or any other reasonable mobile electronic device.

In some embodiments, the illustrative computer-based systems or platforms of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTR0, SHA-1, SHA-2, Tiger (TTH), WHIRLPOOL, RNGs).

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” may refer to a person who receives data provided by the data or service provider over the Internet in a browser session or may refer to an automated software application which receives the data and stores or processes the data.

The aforementioned examples are, of course, illustrative, and not restrictive.

At least some aspects of the present disclosure will now be described with reference to the following numbered clauses.
Clause 1. A system including:
a processor configured to:
   identify a set of copolymers, each copolymer including a chain length and composition;
   analyze the set of copolymers, and determining a set of features of the set of copolymers, the determined set of features at least corresponding to a chain length and composition of monomers;
   perform a copolymer synthesis for each of the set of copolymers based on the determined set of features;
   determine a copolymer complexation based on the performed copolymer synthesis;
   determine a thermostability of a protein based on the determined copolymer complexation;
   execute a regression model, the execution of the regression model being based on the thermostability of the protein;
   execute an optimization model, the optimization model generating a copolymer design, the copolymer design including at least a chain length and composition of monomers of a copolymer that enables the thermostability of the protein; and
   apply a selected copolymer to the protein, where the selected copolymer includes a chain length and composition of monomers corresponding to the copolymer design.
   Clause 2. The system of clause 1, where the thermostability corresponds to the protein being stabilized to retain an activity at a predefined temperature over a predefined time interval, where the predefined temperature may be 37 degrees Celsius; and where the predefined time interval may correspond to 0-168 hours, where the copolymer design further corresponds to a determined retained enzyme activity (REA) for the predefined temperature over the predefined time interval.
   Clause 3. The system of clause 1, where the copolymer synthesis may be performed via execution of an automated PET-RAFT, where the regression model may be a Gaussian regression model, where the optimization model includes an Bayesian optimization.
   Clause 4. The system of clause 1, where the protein is Chondroitinase ABC (ChABC), where the composition of monomers that causes ChABC to have a highest activity is 23.1 mol % of 2-Diethylamino ethyl methacrylate (DEAEMA), 33.2 mol % of BMA, 31.7 mol % of Poly(ethyleneglycol) (n) monomethyl ether monomethacrylate (PEGMA), and 12.0 mol % of [2-(Methacryloyloxy)ethyl]trimethylammonium chloride solution (TMAEMA). In some embodiments, the highest activity may be in a range of +/1 5 mol %, wherein a sum of all mol % may be 100%.
   Clause 5. The system of clause 1, where the monomers are selected from the group consisting of: 2-Diethylamino ethyl methacrylate (DEAEMA), Hydroxypropyl methacrylate (2-HPMA), [2-(Methacryloyloxy)ethyl]trimethylammonium chloride solution (TMAEMA), N-[3-(Dimethylamino)propyl]methacrylamide (DMAPMA), Methyl methacrylate (MMA), 3-Sulfopropyl methacrylate potassium salt (SPMA), Butyl methacrylate (BMA), and Poly(ethyleneglycol) (n) monomethyl ether monomethacrylate (PEGMA). See, e.g., FIG. 3B for further non-limiting examples.
   Clause 6. A composition, including:
a protein; and
a plurality of co-polymers;
   where each co-polymer in the plurality of co-polymers has a specific chain length of monomers, a specific composition of the monomers, or both to stabilize the protein so as to cause the protein to retain an activity at a predefined temperature over a predefined time interval.
   Clause 7. The composition of clause 6, where the monomers are selected from the group consisting of: 2-Diethylamino ethyl methacrylate (DEAEMA), Hydroxypropyl methacrylate (2-HPMA), [2-(Methacryloyloxy)ethyl]trimethylammonium chloride solution (TMAEMA), N-[3-(Dimethylamino)propyl]methacrylamide (DMAPMA), Methyl methacrylate (MMA), 3-Sulfopropyl methacrylate potassium salt (SPMA), Butyl methacrylate (BMA), and Poly(ethyleneglycol) (n) monomethyl ether monomethacrylate.
   Clause 8. The composition of clause 6, where the protein is Chondroitinase ABC (ChABC).
   Clause 9. The composition of clause 8, where the specific composition of monomers that causes ChABC to have a highest activity may be at least 23 mol % of DEAMA, 33 mol % of BMA, 31 mol % of PEGMA, and 12 mol % of TMAEMA. In some embodiments, an activity (e.g., highest) may be 0-100 mol % DEAMA, 0-100 mol % of BMA, 0-100 mol % of PEGMA, and 0-100 mol % of TMAEMA.
   Clause 10. The composition of clause 8, where the predefined temperature may be 37 degrees Celsius; and where the predefined time interval may correspond to 0-168 hours.

Publications cited throughout this document are hereby incorporated by reference in their entirety. While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein may be utilized in any combination with each other. Further still, the various steps may be carried out in any desired order (and any desired steps may be added and/or any desired steps may be eliminated).

What is claimed is:

1. A method comprising:
identifying, by a device, a set of copolymers, each copolymer comprising a chain length and composition;
analyzing, by the device, the set of copolymers, and determining a set of features of the set of copolymers, the determined set of features at least corresponding to a chain length and composition of monomers;
performing, by the device, a copolymer synthesis for each of the set of copolymers based on the determined set of features;
determining, by the device, a copolymer complexation based on the performed copolymer synthesis;
determining, by the device, a thermostability of a protein based on the determined copolymer complexation;
executing, by the device, a regression model, the execution of the regression model being based on the thermostability of the protein;
executing, by the device, an optimization model, the optimization model generating a copolymer design, the copolymer design comprising at least a chain length and composition of monomers of a copolymer that enables the thermostability of the protein; and
applying, by the device, a selected copolymer to the protein, wherein the selected copolymer comprises a chain length and composition of monomers corresponding to the copolymer design.

2. The method of claim 1, wherein the thermostability corresponds to the protein being stabilized to retain an activity at a predefined temperature over a predefined time interval.

3. The method of claim 2, wherein the predefined temperature is 37 degrees Celsius;
and wherein the predefined time interval is corresponds to a range of 0-168 hours.

4. The method of claim 2, wherein the copolymer design further corresponds to a determined retained enzyme activity (REA) for the predefined temperature over the predefined time interval.

5. The method of claim 1, wherein the copolymer synthesis is performed via the device executing an automated PET-RAFT.

6. The method of claim 1, wherein the regression model is a Gaussian regression model.

7. The method of claim 1, wherein the optimization model comprises an Bayesian optimization.

8. The method of claim 1, wherein the protein is Chondroitinase ABC (ChABC).

9. The method of claim 8, wherein the composition of monomers that causes ChABC to have a highest activity is 23.1 mol % of 2-Diethylamino ethyl methacrylate (DEAEMA), 33.2 mol % of BMA, 31.7 mol % of Poly(ethyleneglycol) (n) monomethyl ether monomethacrylate (PEGMA), and 12.0 mol % of [2-(Methacryloyloxy)ethyl]trimethylammonium chloride solution (TMAEMA).

10. The method of claim 1, wherein the monomers are selected from the group consisting of: 2-Diethylamino ethyl methacrylate (DEAEMA), Hydroxypropyl methacrylate (2-HPMA), [2-(Methacryloyloxy)ethyl]trimethylammonium chloride solution (TMAEMA), N-[3-(Dimethylamino)propyl]methacrylamide (DMAPMA), Methyl methacrylate (MMA), 3-Sulfopropyl methacrylate potassium salt (SPMA), Butyl methacrylate (BMA), and Poly(ethyleneglycol) (n) monomethyl ether monomethacrylate (PEGMA).

11. A system comprising:
a processor configured to:
identify a set of copolymers, each copolymer comprising a chain length and composition;
analyze the set of copolymers, and determining a set of features of the set of copolymers, the determined set of features at least corresponding to a chain length and composition of monomers;
perform a copolymer synthesis for each of the set of copolymers based on the determined set of features;
determine a copolymer complexation based on the performed copolymer synthesis;
determine a thermostability of a protein based on the determined copolymer complexation;
execute a regression model, the execution of the regression model being based on the thermostability of the protein;
execute an optimization model, the optimization model generating a copolymer design, the copolymer design comprising at least a chain length and composition of monomers of a copolymer that enables the thermostability of the protein; and
apply a selected copolymer to the protein, wherein the selected copolymer comprises a chain length and composition of monomers corresponding to the copolymer design.

12. The system of claim 11, wherein the thermostability corresponds to the protein being stabilized to retain an activity at a predefined temperature over a predefined time interval, wherein the predefined temperature is 37 degrees Celsius; and wherein the predefined time interval corresponds to a range of 0-168 hours, wherein the copolymer design further corresponds to a determined retained enzyme activity (REA) for the predefined temperature over the predefined time interval.

13. The system of claim 11, wherein the copolymer synthesis is performed via execution of an automated PET-RAFT, wherein the regression model is a Gaussian regression model, wherein the optimization model comprises an Bayesian optimization.

14. The system of claim 11, wherein the protein is Chondroitinase ABC (ChABC), wherein the composition of monomers that causes ChABC to have a highest activity is 23.1 mol % of 2-Diethylamino ethyl methacrylate (DEAEMA), 33.2 mol % of BMA, 31.7 mol % of Poly(ethyleneglycol) (n) monomethyl ether monomethacrylate (PEGMA), and 12.0 mol % of [2-(Methacryloyloxy)ethyl]trimethylammonium chloride solution (TMAEMA).

15. The system of claim 11, wherein the monomers are selected from the group consisting of: 2-Diethylamino ethyl methacrylate (DEAEMA), Hydroxypropyl methacrylate (2-HPMA), [2-(Methacryloyloxy)ethyl]trimethylammonium chloride solution (TMAEMA), N-[3-(Dimethylamino) propyl]methacrylamide (DMAPMA), Methyl methacrylate (MMA), 3-Sulfopropyl methacrylate potassium salt (SPMA), Butyl methacrylate (BMA), and Poly(ethyleneglycol) (n) monomethyl ether monomethacrylate (PEGMA).

16. A composition, comprising:
a protein; and
a plurality of co-polymers;
wherein each co-polymer in the plurality of co-polymers has a specific chain length of monomers, a specific composition of the monomers, or both to stabilize the protein so as to cause the protein to retain an activity at a predefined temperature over a predefined time interval.

17. The composition of claim 16, wherein the monomers are selected from the group consisting of: 2-Diethylamino ethyl methacrylate (DEAEMA), Hydroxypropyl methacrylate (2-HPMA), [2-(Methacryloyloxy)ethyl]trimethylammonium chloride solution (TMAEMA), N-[3-(Dimethylamino)propyl]methacrylamide (DMAPMA), Methyl methacrylate (MMA), 3-Sulfopropyl methacrylate potassium salt (SPMA), Butyl methacrylate (BMA), and Poly(ethyleneglycol) (n) monomethyl ether monomethacrylate (PEGMA).

18. The composition of claim 16, wherein the protein is Chondroitinase ABC (ChABC).

19. The composition of claim 18, wherein the composition of monomers that causes ChABC to have a highest activity is 23.1 mol % of 2-Diethylamino ethyl methacrylate (DEAEMA), 33.2 mol % of BMA, 31.7 mol % of Poly(ethyleneglycol) (n) monomethyl ether monomethacrylate (PEGMA), and 12.0 mol % of [2-(Methacryloyloxy)ethyl]trimethylammonium chloride solution (TMAEMA).

20. The composition of claim 18, wherein the predefined temperature is 37 degrees Celsius; and wherein the predefined time interval corresponds to a range of 0-168 hours.
